(19) United States
(12) Patent Application Publication
(10) Pub. No.: US 2004/0171045 A1
(43) Pub. Date: Sep. 2, 2004

(54) Assay for predicting cell activity

(75) Inventors: Joost Dick de Bruijn, Amersfoort (NL); Sandra Claudia da Silva Madureira Mendes, Gouda (NL); Clemens Antoni van Blitterswijk, Hekendorp (NL)

Correspondence Address: BANNER & WITCOFF, LTD., 28 STATE STREET, 28th FLOOR, BOSTON, MA, 02109-9601, US

(73) Assignee: IsoTis N.V., Bilthoven (NL)

(21) Appl. No.: 10/730,770

(22) Filed: Dec. 8, 2003

Related U.S. Application Data
(63) Continuation of application No. PCT/NL02/00371, filed on Jun. 7, 2002.

(30) Foreign Application Priority Data
Jun. 8, 2001 (EP) 01202220.8

(51) Int. Cl. C12Q 1/68; G01N 33/53; G01N 33/567; C12N 5/08

(52) U.S. Cl. 435/6; 435/7.2; 435/366

(57) ABSTRACT

The invention relates to a method of determining in vitro the capacity of a cell population to induce bone formation in vivo comprising the steps of: a) providing a sample of a cell population; b) dividing said sample into a first and a second part containing an equal number of cells; c) culturing the first part in the presence of an osteogenic stimulation factor; d) culturing the second part in the absence of an osteogenic stimulation factor; e) determining degrees of expression of a bone-specific protein; and f) comparing the degrees of expression of the bone-specific protein of the first part and the second part thereby providing a measure for the capacity of the bone cell population to induce bone formation in vivo.

The invention relates to an in vitro method for determining the capacity of cells to induce bone formation in vivo. The invention further relates to a kit comprising the means to carry out said method.

The increasing demand for organ and tissue transplants has motivated several universities and private companies to develop research in the field of tissue engineering. At present, and with regard to the repair of skeletal defects, numerous investigators have purposed the use of autologous cultured tissue approaches as an alternative to the traditional bone grafting therapies. The engineering of bone tissue is based on the idea of seeding a suitable implant material with a patient's own cells that, during in vitro culture and prior to transplantation into the defect site, will form a bone tissue coating over the material surface.

The bone marrow stromal cell population is known to contain progenitors capable of differentiation into the mesenchymal lineages of bone, cartilage, fat and other connective tissues. Therefore, they constitute an interesting population for use in cell therapies. Furthermore, bone marrow stromal cells can be easily isolated from the patients marrow, extensively expanded during in vitro culture and, finally, induced to further differentiate into the relevant lineage. The in vitro and in vivo osteogenic potential of adult human bone marrow stromal cells (HBMSC) cultured on a porous calcium phosphate material has already been reported.

However, in several of these studies, in vivo bone induction by HBMSC cultures did not occur in all of the tested patients. Moreover, osteoinductivity of the cultures was found to decrease with patient age. Therefore, the development of an analysis method that will allow predicting in vitro, and in the early stages of proliferation, the performance of the tissue-engineered implants in an in vivo situation is of extreme importance.

The international patent application 94/26872 describes a method for assessing bone cell activity, wherein bone cells are cultured in vitro on a thin calcium phosphate film. However, this method merely provides information on the resoiptive activity of osteoclasts. It is not aimed at resulting in a determination of the capacity of bone cells taken in a biopsy to induce de novo bone formation in vivo after implantation in the form of a tissue engineered bone construct. Moreover, the method involves a lengthy and cumbersome procedure.

The present invention aims to provide a method by which the capacity of bone cells to induce bone formation in vivo can be determined in vitro. It is desired that the objective method is sufficiently simple to perform and has a sufficient level of sensitivity.

Surprisingly, it has been found that a relationship exists between the effect of an osteogenic stimulation factor, such as dexamethasone (dex) or vitamin D3, on the expression of a bone-specific protein by a bone cell population and the capacity of the bone cell population to induce bone formation in vivo. Accordingly, the desired determination of osteogenic potential can be achieved by comparing the expression of the bone-specific protein by bone cells cultured in the presence of dex with said expression by bone cells cultured without dex.

Thus, the invention specifically relates to a method for determining in vitro the capacity of a cell population to induce bone formation in vivo comprising the steps of:

a) providing a sample of a cell population;

b) dividing said sample into a first and a second part containing an equal number of cells;

c) culturing the first part in the presence of an osteogenic stimulation factor;

d) culturing the second part in the absence of an osteogenic stimulation factor;

e) determining degrees of expression of a bone-specific protein; and

f) comparing the degrees of expression of the bone-specific protein of the first part and the second part thereby providing a measure for the capacity of the bone cell population to induce bone formation in vivo.

A method according to the invention allows detection of cultures with low osteoinductivity, indicating the need for a second biopsy procedure or making possible further enhancement of the bone forming capacity of the cultures through the use of bone growth factors, such as bone morphogenetic proteins, that are known to posses a strong stimulatory effect on osteogenic commitment and differentiation of HBMSC. Furthermore, the present method can be used as a quality control step prior to transplantation of the implant into a patient defect site. A method according to the invention, therefore, reduces the frequency of cases in which bone induction in vivo is not achieved, thereby preventing failures of surgical procedures for bone implantations or substitutions.

The cell population of which the osteogenic potential can be determined in a method according to the invention can in principle be any sample of cells that are at least to some extent capable of undergoing differentiation to bone cells, such as osteoblasts and/or osteoclasts. Preferably, the bone cell population comprises human bone marrow stromal cells and/or human osteoprogenitor cells.

It is preferred that the sample of the cell population is obtained through a biopsy from a patient who has to undergo surgery for implantation of a bone prostheses or construct. The biopsy may be taken by any conventional means. Suitable locations for taking the biopsy are the iliac crest, the spine, the mandibula, and the acetobular fossa.

It is possible that the cell population to be investigated has undergone one or more culturing steps prior to carrying out a method according to the invention. This pre-culturing can be advantageous in order to obtain a sufficient number of cells, which means that the culturing mainly comprises proliferation of the cells. Another goal of a pre-culturing may be to enhance adhesion of the cells to a substrate used in culturing. Thus, it is possible that the cells have undergone some degree of differentiation before the present method is carried out. A method according to the invention may be performed on cell populations of varying degrees of differentiation, as long as not all cells are fully differentiated into osteocytes. Preferably, the method is performed in an early differentiation stage, i.e. in the osteoprogenitor stage.

To perform a method according to the invention, the sample is divided into two equal parts. This means that both parts should contain substantially the same number of cells, which can be achieved and checked by methods well-known to the skilled person.

Both parts are cultured in a suitable culture medium. A suitable culture medium may be based on Dulbecco's alpha Minimal Essential Medium (α-MEM), or any other conventional suitable medium. Preferably, the medium contains additional amounts of L-ascorbic acid 2-phosphate, an antibiotic, serum, and/or a growth factor. The antibiotic is preferably chosen from the group of penicillin G, gentamicin, fungizone, and streptomycin. The growth factor is preferably basic fibroblast growth factor (bFGF). A highly preferred culture medium is a medium as described in the international patent application WO-A-01/48147, of which the contents are incorporated herein by reference.

Since it is an important aspect of the invention that an effect of an osteogenic stimulation factor on the expression of alkaline phosphatase by the bone cell population is determined, one of the two parts of the sample of the bone cell population is cultured in the presence of an osteogenic stimulation factor, while the other is cultured without said factor. In this regard, an osteogenic stimulation factor is an agent that can induce cells to differentiate into bone cells or can induce cells to produce bone-specific proteins. Preferred osteogenic stimulation factors are dexamethasone (dex) or vitamin D3. These are preferably used in an amount ranging from 10⁻¹⁰ to 10⁻⁵ M.

During culturing the cells will or will not express a certain amount of a bone-specific protein. Preferably, the bone-specific protein is chosen from the group of alkaline phosphatase, osteocalcine, bone sialo protein, osteopontine and osteonectine. More preferably, the bone-specific protein is alkaline phosphatase (ALP) or osteocalcine. In case the bone-specific protein is ALP, dexamethasone is preferably used as osteogenic stimulation factor. In case the bone-specific protein is osteocalcine, vitamin D3 is preferably used as osteogenic stimulation factor.

Culturing of the cells is performed for a sufficient length to allow cell to express the bone-specific protein. In principle, the duration of the culturing can suitably be chosen by the skilled person based on his experience to obtain a large enough number of cells and to allow sufficient expression of the bone-specific protein. Nevertheless, it is noted that, depending on the cell population and the circumstances, it is not always necessary that the duration of culturing is at least one doubling time of the cell population. It is preferred however, that culturing takes place for two to fifteen doubling times.

In accordance with the invention, the expressed amount of the bone-specific protein is determined. This can be done in various ways, depending on the type of bone-specific protein.

In a preferred embodiment, the bone-specific protein is ALP, which may be detected by labelling with an antibody specific for ALP and detecting the antibodies. Thus, the cells of both parts are contacted with the antibody. A preferred example of a suitable antibody is anti-ALP (hybridoma B4-78). The labelling of the cells with the antibody may be carried out in any conventional manner. A suitable manner to determine the degree of expression of ALP of both parts of the sample of the bone cell population is by flow cytometry. The expression of ALP is measured as the percentage of ALP positive cells as compared to the total cell population.

It is also possible to detect ALP by allowing it to convert a substrate for the enzyme ALP and detecting formed reaction product. Suitable substratea in this respect are para-nitro phenyl phosphate and alpha-naphtol AS-BL phosphate. Hydrolysis of the latter leads to the formation of a highly insoluble naphtol that may be coupled to a suitable diazonium salt that it preferably present. A suitable example of such a diazonium salt is fast blue RR. The coupling provokes a colour reaction that can be detected by the naked eye or by UV. The reaction product obtained after conversion of para-nitro phenyl phosphate by ALP may be detected using Sigma 104R phosphatase substrate and UV. When the substrate is para-nitro phenyl phosphate, the cells are preferably first subjected to lysis and sonification.

In another preferred embodiment, the bone-specific protein is osteocalcine, of which the expression is preferably triggered by addition of vitamin D3. The degree of expression of osteocalcine may be detected by labelling with an antibody specific for osteocalcine and detecting the antibodies. Thus, the cells of both parts are contacted with the antibody. A preferred example of a suitable antibody is mouse anti-human antibody (Zymed). The labelling of the cells with the antibody may be carried out in any conventional manner. A preferred manner to determine the degree of expression of osteocalcine of both parts of the sample of the bone cell population in accordance with this embodiment is by UV or flow cytometry. The expression of osteocalcine is measured as the percentage of osteocalcine positive cells as compared to the total cell population.

In accordance with the invention it has been found that the relationship between the degrees of expression of the bone-specific protein of both parts provides a prediction of the capacity of the bone forming potential of the cultured cells. Statistical analysis has indicated that this capacity may be expressed in the form of the logarithm of the ratio of the degrees of expression of the bone-specific protein of the first part, cultured in the presence of osteogenic stimulation factor, and the second part, cultured without osteogenic stimulation factor. Thus, a quantitative measure is obtained for the osteogenic potential of a cell population.

In order to determine whether the cell population is suitable to use in tissue engineering a medical implant, such as a bone construct, the obtained value may be compared to a discriminating index. The index can be obtained from statistical analysis from a significant number of experiments and has been found to lie between 0.17 and 0.23. If the value calculated for the logarithm of the ratio of the degrees of expression of the bone-specific protein of the first and second parts is larger than the index, it may be assumed that the osteogenic potential of the cell population is sufficient to use said population in tissue engineering.

The invention further relates to a kit for carrying out the above described method. Said kit comprises means to provide a sample of a bone cell population, means for culturing bone cells, an osteogenic stimulation factor, and means for detecting a bone-specific protein. The means to provide a sample of a bone cell population preferably comprise means to take a biopsy, and the means for culturing bone cells preferably comprise a suitable culture medium. The means for detecting a bone-specific protein will depend on the nature of the bone-specific protein. Preferably, said means comprise an antibody or a substrate for the bone-specific protein, and optionally also means for detecting said antibody or the reaction product of conversion of the substrate by the bone-specific protein.

The invention will now be further elucidated by the following, non-restrictive examples.

### EXAMPLES

Materials and Methods

Human Bone Marrow Stromal Cell (HBMSC) Harvest and Culture

Bone marrow aspirates (10-30 ml) were obtained from 14 patients that had given written informed consent. Donor information is summarized in Table 1.

TABLE 1
HBMSC donor information

| | Source of bone | | | Identification |
|---|---|---|---|---|
| Donor | marrow | Gender | Age | number |
| 1 | Iliac crest | M | 75 | U00079 |
| 2 | Acetabular fossa | M | 86 | U00084 |
| 3 | Iliac crest | M | 74 | U00169 |
| 4 | Iliac crest | M | 45 | U00182-2 |
| 5 | Iliac crest | F | 39 | U00212 |
| 6 | Acetabular fossa | F | 54 | U00230 |
| 7 | Spine | M | 44 | U00106 |
| 8 | Iliac crest | F | 69 | U00173 |
| 9 | Iliac crest | M | 74 | U00174 |
| 10 | Acetabular fossa | F | 72 | U00178 |
| 11 | Iliac crest | F | 70 | U00179 |
| 12 | Iliac crest | F | 74 | U00090 |
| 13 | Acetabular fossa | F | 67 | U00180 |
| 14 | Spine | M | 44 | U00091 |

F = female
M = male

The bone marrow specimens were collected in heparinized tubes and transported at room temperature. Cells were re-suspended with a 20G needle, plated at a density of 500,000 nucleated cells/cm² and cultured in minimum essential medium (α-MEM, Life Technologies, The Netherlands) containing 10% foetal bovine serum (FBS, Life Technologies, The Netherlands), antibiotics (AB), 0.2 mM L-ascorbic acid 2-phosphate (AsAP, Life Technologies, The Netherlands) and 1 ng/ml basic fibroblast growth factor (bFGF, Instruchemie, The Netherlands). Cells were grown at 37° C. and in a humid atmosphere with 5% CO₂. The culture medium was refreshed twice a week and, at near confluence, the adherent cells were washed with phosphate buffered saline solution (PBS, Life Technologies, The Netherlands) and enzymatically released by means of a 0.25% trypsin—EDTA solution (Sigma, The Netherlands). Cells were plated at a density of 5,000 cells per cm² and subsequent passages were performed when cells were near confluence (80-90%).

Scaffold Material

Porous granules of coraline hydroxyapatite (HA) with an average surface area of 0.2-0.3 cm² were used as scaffold material. The interconnected pores had a median diameter of 435 μm and the size of the particles was approximately 3×2×2 mm.

Antibodies

The purified anti-ALP (hybridoma B4-78), anti-PCI (M-38) and anti-OP (MPIIIB10) were obtained from the Developmental Studies Hybridoma Bank (University of Iowa, USA). The control mouse immunoglubin G (IgG2a) monoclonal antibody and the secondary antibody goat anti-mouse IgGγ-chain-specific-FITC were purchased from Dako (Denmark).

Temporal Expression of ALP

Fourth passage HBMSC (donors 1 to 14) were plated at a density of 5,000 cells per cm² and cultured for 8 to 9 days both ) in two different types of media: (i) α-MEM containing 10% FBS, AB, 0.2 mM AsAP and 0.01M β-glycerophosphate (βGP, Sigma, The Netherlands) (control medium) and (ii) control medium with the addition of 10⁻⁸ M dexamethasone (dex, Sigma, The Netherlands) (+dex medium). The expression of ALP was evaluated by flow cytometry at several culture periods (from day 1 to day 9, three to four measurements were performed for each culture). Briefly, after trypsinisation, cells were washed twice in wash buffer and blocked against non-specific binding (see above). Cells (approx. 0.1-0.3E6/staining) were then resuspended in blocking buffer containing: (a) control mouse anti-human IgG2a (1:5 dilution) and (b) ALP monoclonal antibody (1:10 dilution). After incubation on ice for 45 minutes and washing, antibody reactivity was detected by suspending the cells with blocking buffer containing goat anti-mouse IgGγ-chain-specific-FITC (1:5 dilution). Cells were incubated on ice and in the dark for 30 minutes. After washing, the cells were resuspended in 200 μl of FACS-flow/staining and analyzed using a FACS Calibur apparatus (Becton Dickinson Immunocytometry systems). For each measurement 10,000 events were collected.

Enzymatic Alkaline Phosphatase Staining

Human bone marrow stromal cells were plated at a density of 5000 cells per cm(superscript: 2) in 6 wells plates (n=3) and cultured with and without dexamethasone for 7 days. After washing with PBS they were fixated with 4% paraformaldehyde in Sorensenbuffer for 4-6 hours and afterwards rinsed 2-3 times with distilled water. Fast blue RR salt (Sigma) was dissolved in Naphtol As-BI stock solution (1 mg/ml) and filtered. The samples were placed in the staining solution and incubated at room temperature for 10-60 minutes until the staining was visible. Afterwards the samples were washed with distilled water.

Alkaline Phosphatase Replating

Human bone marrow stromal cells were plated at a density of 5000 cells per cm(superscript: 2) in 6 wells plates (n=3) and cultured with and without dexamethasone for 7 days. After washing with PBS they were subjected to lysis and sonification. As substrate for ALP, para-nitro phenyl phosphate (PNP) was used (10 mM PNP in 1 ml diethanol amine and 1 mM magnesium chloride (MgCl₂.6H₂O) at pH 9.8). The reaction product was detected using Sigma 104R phosphatase substrate (52.6 mg) dissolved in 10 ml ALP buffer (10 ml 10M diethanol amine and 90 ml demi-water to which, after overnight incubation, 10.33 mg magnesium chloride (MgCl₂.6H₂O) was added). The pH was adjusted to 9.8 using 1N HCl. 100 μl of this substrate was added to 100 μl of the cell lysate for 15 minutes at 37° C. A yellow colour change was observed in a plate reader at 405 nm. The results were calibrated and adjusted for the amount of DNA in a known manner.

Osteocalcin Detection: Flow Cytometry

Human bone marrow stromal cells were seeded at a density of 5000 cells per cm(superscript: 2) in T75 flask and cultured for 3-7 days in media with and without 1.25(OH)D3 (vitamin D3). After trypsinisation the cells were blocked against nonspecific binding. Cells were then resuspended in FIX solution A (Caltag laboratories) incubated for 15 minutes and washed. A mixture 1:1 was made of PERM solution B (Caltag laboratories) and the osteocalcin mouse anti-human antibody (Zymed) dissolved in the blocking buffer (1:100 dilution). The cells were incubated for 15 minutes with the mixture. After incubation and washing, antibody reactivity was detected by suspending the cells with blocking buffer containing Goat anti-mouse FITC conjugated F (ab′)2 fragment (DAKO) (1:100 dilution) and incubating the cells for 30 minutes in the dark. After washing the cells were resuspended in FACS flow/staining and analyzed by a FACS Calibur apparatus (Becton Dickinson Immunocytometry systems). For each event 10.000 events were collected.

Osteocalcin Detection: Immunostainings

Human bone marrow stromal cells were seeded at a density of 5,000 cells per cm(superscript: 2) in tissue culture chamber slides and cultured for a minimum of 3 days in media with and without 1.25(OH)D3 (vitamin D3). The staining procedure for the slides is as described above for flowcytometry. After the last incubation step the cells are washed and detection of osteocalcin can be observed trough a microscope with UV-light. As a control for both the flowcytometry and the immunostainings the SOAS-2 cell line (DSMZ) was used.

In Vivo Osteogenic Potential of HBMSC

HBMSC (passage 4, donor 1 to 14) were seeded on porous HA granules, at a density of 200,000 cells/particle and cultured for one week in (+) dex medium. Following this period, and prior to implantation, the tissue engineered samples were soaked in serum free medium and washed in phosphate buffered solution pre-warmed to 37° C. Samples (n=6 per donor) were then implanted into subcutaneous pockets created in the back of immunodeficient mice. Samples of each culture were divided over two animals. At the end of the six-week survival period, the implants were removed and fixed in 1.5% glutaraldehyde in 0.14 M cacodylic acid buffer, pH 7.3. The fixed samples were dehydrated and embedded in methyl methacrylate. The sections were processed undecalcified on a histological diamond saw (Leiden microtome cutting system) and then stained with basic fuchsin and methylene blue in order to detect bone formation.

Statistics

Statistical analysis was performed using both t student tests and Mann-Whitney U tests assuming non equal variances. Statistical significance was defined as p<0.05. When calculating an index to predict osteogenic capacity, an asymptotic curve test was also performed to analyse statistical significance between the ROC curve and the curve of no discrimination.

Results

Temporal Expression of ALP

In HBMSC cultures from each donor, the temporal evolution pattern of ALP+cells was similar in both culture conditions. However, in cultures treated with dex, the fraction of ALP+cells was consistently higher as compared to control cultures (FIG. 1a). Statistical analysis revealed that after the first two days of culture, the proportion of ALP+cells in the (+) dex condition was significantly higher as compared to the control (p<0.05), revealing that dex stimulation induced an increase in the fraction of committed osteoprogenitor cells. In the majority of the donors tested (12 of 14), the relative amount of ALP+cells increased during culture period reaching a maximum value and decreased thereafter. The time period required to achieve the maximum of ALP expression, as well as the value of the maximal fraction of ALP+cells, was affected by the culture conditions and markedly donor dependent (FIG. 1a and b). In HBMSC cultures from 2 of the 14 patients, the percentage of cells expressing ALP was above 80% in the beginning of the culture and decrease thereafter (data not shown).

Quantification of Osteoprogenitor Cells in Culture

An approach that may allow for the indirect quantification of osteoprogenitor cells is the degree of culture stimulation by dex with regard to the fraction of ALP+cells. That is, cultures exhibiting a high fold increase in the amount of cells expressing ALP due to dex treatment most likely contain a higher proportion of osteoprogenitor cells as compared to cultures in which stimulation by dex induces a lower fold increase in ALP expression. Therefore, for each donor and culture period, the degree of stimulation by dex was measured through the ratio between the fraction of ALP+cells in the (+) dex and control conditions. Both t student and Mann-Whitney U tests indicated that, after the first two days in culture, this ratio was time independent, revealing that the optimal cell response to dex treatment occurred after the first 48 hours. To verify whether the degree of culture response to dex was correlated to the in vivo bone induction ability of the cultures, for each donor the average ratio was determined using the measurements performed from day 3 to day 9 (Table 2). This ratio, taken as an indirect measure for the proportion of osteoprogenitor cells, was then compared to the in vivo osteogenic potential of the cultures.

TABLE 2
Degree of dex stimulation measured as the ratio between
the fraction of ALP⁺ cells in the (+) dex and control conditions.

| Donor | Ratio | Identification number |
|---|---|---|
| 1 | 1.53 | U00079 |
| 2 | 1.53 | U00084 |
| 3 | 1.40 | U00169 |
| 4 | 2.71 | U00182-2 |
| 5 | 2.53 | U00212 |
| 6 | 1.52 | U00230 |
| 7 | 1.25 | U00106 |
| 8 | 3.72 | U00173 |
| 9 | 1.56 | U00174 |
| 10 | 2.12 | U00178 |
| 11 | 2.40 | U00179 |
| 12 | 2.56 | U00090 |
| 13 | 1.55 | U00180 |
| 14 | 1.80 | U00091 |

In Vivo Osteogenic Potential of HBMSC

To determine the in vivo bone inducing ability of HBMSC cultures, cells were seeded into porous HA, at a density of 200,000 cells/particle and further cultured for one week, in the presence of dex. Following this period, the samples were implanted under the skin of immunodeficient mice. Six weeks post implantation, de novo formed bone was found in all the samples from 8 of the 14 assessed donors (1-2, 4-5, 8, 11-12, 14). Woven, mineralized bone tissue was observed in direct contact with the ceramic material, indicating that the implanted cells survived and further differentiated into osteoblasts. The bone matrix displayed embedded osteocytes and blood vessels were often observed close to the newly deposited bone. The HBMSC cultures from these donors revealed a good agreement between the in vivo and vitro data, in which the osteogenic character of the cultures was demonstrated by the expression of PCI, OP (donor 1, 2, 4, 5) and by an increase in ALP expression after treatment with dex. However, and although in vitro testing also indicated expression of PCI, OP (donor 3, 6, 7) and an increase in ALP expression after treatment with dex, HBMSC cultures from donors 3, 6, 7, 9, 10 and 13 failed to induce in vivo osteogenesis.

In Vivo Osteogenic Potential Versus Degree of Stimulation by Dex with Regard to ALP Expression

The in vivo bone formation capacity of HBMSC could not be related to their in vitro expression of PCI, OP or ALP. However, the relative increase in the proportion of ALP+cells in culture following dex treatment proved to be related to the in vivo bone induction capacity of the cultures, revealing that these relative increase, expressed by the ratio between the fraction of ALP+cells in (+) dex and control conditions, can be taken as an indirect measurement for the proportion of osteoprogenitor cells in culture. Our data demonstrated that the degree of dex stimulation was higher in bone forming cultures as compared to cultures that failed to induce osteogenesis (FIG. 2). Both t student and Mann-Whitney U tests revealed a statistically significant difference between bone forming and non bone forming cultures with regard to the increase on ALP expression after dex treatment (p=0.021, t student test; p=0.029, Mann-Whitney U test).

Following these results, we performed a preliminary attempt to define an index to predict in vitro the in vivo performance of the implant. Statistical analysis indicated that this index should be based on the log of the ratio between the proportion of cells expressing ALP in the (+) dex and control condition. This parameter displayed the smallest variance and the best discrimination in the t test (p=0.016 for log ratio and p=0.021 for ratio). Therefore, these values were calculated for each donor and the best discriminating index was determined using the so called ROC curve (FIG. 3). The test result for the log ratio revealed a statistical significance (p=0.028, asymptotic curve test) between the obtained ROC curve and the curve of no discrimination (FIG. 3). The data further revealed that the best index should be 0.20, meaning that in order to obtain in vivo bone induction by HBMSC log ratio should be equal or higher than 0.20. This index, and for the donor population assessed, provided a correct prediction (sensitivity) in 75% of the cases and an accuracy in classifying non bone forming cultures (specificity) of 83.3%.

### LEGENDS TO FIGURES

FIG. 1—Temporal expression of ALP in HBMSC cultures: Effect of dexamethasone treatment and variance between donors. (a) Donor 11 and (b) Donor 9.

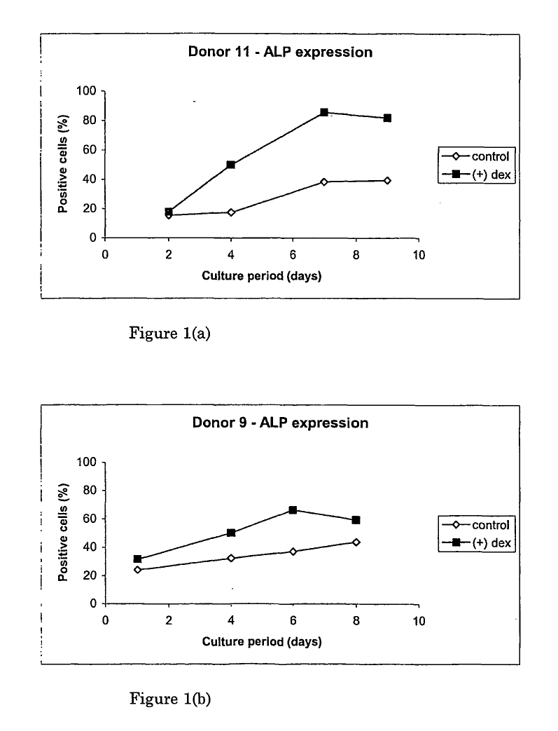

FIG. 2—Relative increase in the fraction of ALP+cells in bone forming and non bone forming cultures, after dex treatment. (⋄) Individual values of 14 donors; (♦) Average of each population; (*) Statistical significance was observed: p=0.021 in t test and p=0.029 in Mann-Whitney U test.

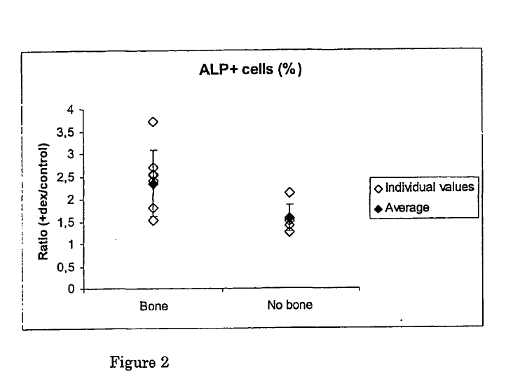

FIG. 3—Determination of the osteoinductive index by a ROC curve analysis. Sensitivity was defined as the ratio between correctly predicted cases and the total number of cases. Specificity was defined as the ratio between correctly predicted non bone forming cases and the total number of cases.

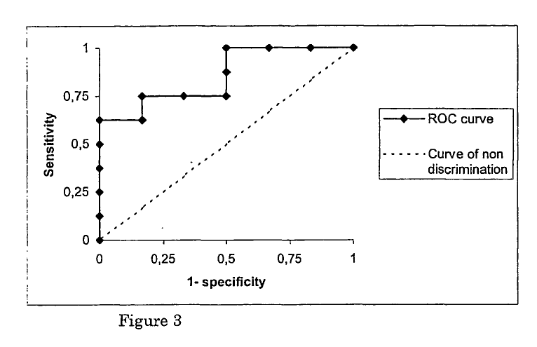

1. A method for determining in vitro the capacity of a cell population to induce bone formation in vivo comprising the steps of:
a) providing a sample of a cell population;
b) dividing said sample into a first and a second part containing an equal number of cells;
c) culturing the first part in the presence of an osteogenic stimulation factor;
d) culturing the second part in the absence of an osteogenic stimulation factor;
e) determining degrees of expression of a bone-specific protein; and
f) comparing the degrees of expression of the bone-specific protein of the first part and the second part thereby providing a measure for the capacity of the bone cell population to induce bone formation in vivo.

2. A method according to claim 1, wherein the sample of a cell population is obtained through a biopsy from a patient who has to undergo surgery to receive a bone implant.

3. A method according to claim 2, wherein the cell population comprises human bone marrow stromal cells, and/or human osteoprogenitor cells.

4. A method according to any of the preceding claims, wherein the osteogenic stimulation factor is dexamethasone or vitamin D3.

5. A method according to claim 4, wherein the osteogenic stimulation factor is used in an amount of 10⁻¹⁰ to 10⁻⁵ M.

6. A method according to any of the preceding claims, wherein the cells are cultured for 2 to 15 doubling times.

7. A method according to any of the preceding claims, wherein the cells are cultured in a culture medium based on α-MEM.

8. A method according to claim 7, wherein the culture medium further comprises L-ascorbic acid 2-phosphate, an antibiotic, serum, and/or a growth factor.

9. A method according to claim 8, wherein the growth factor is basic fibroblast growth factor (bFGF).

10. A method according to claim 8, wherein the antibiotic is chosen from the group of penicillin G, gentamicin, fungizone, and streptomycin.

11. A method according to any of the preceding claims, wherein the bone-specific protein is chosen from the group of alkaline phosphatase, osteocalcine, bone sialo protein, osteopontine and osteonectine.

12. A method according to claim 11, wherein the bone-specific protein is alkaline phosphatase, of which the degree of expression is determined by labeling the cells with an antibody specific for alkaline phosphatase and detecting labeled cells using flow cytometry.

13. A method according to claim 12, wherein the antibody is anti-ALP (hybridoma B4-78).

14. A method according to claim 11, wherein the bone-specific protein is alkaline phosphatase, of which the degree of expression is determined by contacting the cells of the first and second parts to a substrate for alkaline phosphatase, allowing the substrate to be converted to a reaction product, and detecting the reaction product.

15. A method according to claim 14, wherein the substrate is para-nitro phenyl phosphate or alpha-naphtol AS-B1 phosphate.

16. A method according to claim 15, wherein the cells are contacted with alpha-naphtol AS-BL phosphate in the presence of a diazonium salt, preferably fast blue RR.

17. A method according to claim 15, wherein the cells are contacted with para-nitro phenyl phosphate, and the reaction product is reacted further with Sigma 104R phosphatase substrate and subsequently detected by WV.
